(19) United States
(12) Patent Application Publication
(10) Pub. No.: US 2020/0263118 A1
(43) Pub. Date: Aug. 20, 2020

(54) Devices, Methods, And Compositions For Restricting Cell Position And Stabilizing Cells In Culture Systems

(71) Applicant: President and Fellows of Harvard College, Cambridge, MA (US)

(72) Inventors: Kambez Hajipouran Benam, Cambridge, MA (US); Richard Novak, Boston, MA (US); Donald E. Ingber, Boston, MA (US)

(21) Appl. No.: 15/781,080

(22) PCT Filed: Dec. 2, 2016
(86) PCT No.: PCT/US2016/064647

§ 371 Date: Jun. 1, 2018

Related U.S. Application Data
(60) Provisional application No. 62/263,383, filed on Dec. 4, 2015.
(60) Provisional application No. 62/333,007, filed on May 6, 2016.

(51) Int. Cl. C12M 1/12; C12M 3/06; B01L 3/00; A01N 1/02

(52) U.S. Cl. CPC C12M 25/02; C12M 23/16; B01L 2300/06; A01N 1/0242; B01L 2200/10; B01L 3/502715

(57) ABSTRACT

A device is directed to simulating a function of a tissue, and includes a first structure defining a first chamber, a second structure defining a second chamber, and a porous membrane located at an interface region between the first chamber and the second chamber. The membrane has a first side facing toward the first chamber and a second side facing toward the second chamber, the membrane separating the first chamber from the second chamber. The first side includes a fluid-permeable, stimulus-responsive polymer gel thereon, the second side including at least one layer of cells adhered thereon.

### CROSS-REFERENCE TO RELATED APPLICATIONS

This application claims priority to and benefit of U.S. Provisional Patent Application Ser. No. 62/263,383, filed on Dec. 4, 2015, and to U.S. Provisional Patent Application Ser. No. 62/333,007, filed on May 6, 2016, each of which is hereby incorporated by reference herein in its entirety.

### GOVERNMENT SUPPORT

The invention was made with Government Support under W911NF-12-2-0036 awarded by the Defense Advanced Research Projects Agency of the U.S. Department of Defense. The government has certain rights in the invention.

### FIELD OF THE INVENTION

The present invention relates generally to simulation of a tissue function, and, more particularly, to preventing cell invasion in a microfluidic device.

### BACKGROUND OF THE INVENTION

Human endothelial cells have lengths and widths on the micron scale, e.g., in the 5-25 micron (“μm”) range, when flattened within monolayers in vitro, when lining the vasculature, or within engineered organs-on-chips that mimic in vivo tissue and organ architecture. Additional details in reference to the scale of human endothelial cells are described in “Image Analysis of Endothelial Microstructure and Endothelial Cell Dimensions of Human Arteries—A Preliminary Study,” by Bora Garipcan et al., published online on Dec. 10, 2010 (DOI: 10.1002/adem.201080076), which is incorporated herein by reference in its entirety. These cells, similar to many other human cell types, possess great migratory potential that allows them to reconfigure their shape and migrate through small pores (>1 μm diameter) and to move to adjacent environments. Additional details in reference to cell migration are described in “Endothelial Cell Migration During Angiogenesis,” by Laurent Lamalice et al., accepted on Jan. 24, 2007 (DOI: 10.1161/01.RES.0000259593.07661.1e), which is incorporated herein by reference in its entirety.

The cell migration is useful, for example, when simulating angiogenesis or studying immune cell recruitment in vitro. However, this capability can create problems when cells, such as endothelial cells, are cultured on porous membranes within organs-on-chips or other in vitro culture systems. For example, undesired cell migration occurs through the pores and interferes with plating or growth of neighboring tissues, whose presence is required to create critical tissue-tissue interfaces that mimic organ architecture with endothelial cells. This problem can occur when pores are greater than ˜1 μm in diameter. For example, when human umbilical vein endothelial cells (“HUVECs”) are seeded in a microfluidic device, cells can escape from a seeded channel to an opposite channel within 1-3 days of cell seeding. According to this example, the microfluidic device includes two polydimethylsiloxane (“PDMS”) top and bottom slabs sandwiching a porous polyethylene terephthalate (“PET”) or polycarbonate membrane containing 5 μm pores to create a top and a bottom channel. This type of cell migration can lead to experimental failures. In other examples, cell escape occurs when using membranes with 3 μm or 8 μm pores.

To prevent cell migration into another channel, smaller pore size of a member has been previously used. However, using pores that are too small to allow cells to pass through also prevents studies where some cell migration is desirable, such as immune cell migration in mature tissue or when cell-cell contacts between the layers is crucial for function (e.g., podocyte-endothelial cell interactions). Accordingly, there is a need for controllable, reversible, and robust blocking of microfluidic channel regions for restricting cell position and/or stabilizing cells in culture systems.

### SUMMARY OF THE INVENTION

According to one embodiment of the present invention, a device includes a first structure defining a first chamber, the first chamber including cells adhered thereon, and the first chamber further including a stimulus-responsive polymer gel on top of the cells.

According to one aspect of the device described above, the device is a microfluidic device, and the first chamber includes a first microfluidic channel.

According to another aspect of the device described above, the microfluidic device further includes a second microfluidic channel in fluidic communication with the first microfluidic channel.

According to yet another aspect of the device described above, the first chamber includes an agent that is blocked from contacting the cells by the gel.

According to yet another aspect of the device described above, the agent is a drug.

According to yet another aspect of the device described above, the agent is a growth factor.

According to yet another aspect of the device described above, the gel prevents the cells from differentiating.

According to yet another aspect of the device described above, the device is housed in a storage container.

According to yet another aspect of the device described above, the gel is fluid permeable.

According to yet another aspect of the device described above, the gel is water impermeable.

According to yet another aspect of the device described above, the device further includes a porous membrane located at an interface region between the first microfluidic channel and the second microfluidic channel, the membrane having a first side and a second side the cells being adhered on the first side.

According to yet another aspect of the device described above, the stimulus-responsive polymer gel is capable of being dissolved by exposure to an external stimulus.

According to yet another aspect of the device described above, the external stimulus is selected from a group consisting of temperature, light, pH, a small molecule, an enzyme and a combination of two or more thereof.

According to yet another aspect of the device described above, the fluid-permeable, stimulus-responsive polymer gel is selected from a group consisting of Pluronic F127, Pluronic F68, poly(N-isopropylacrylamide), alginates, and a combination of two or more thereof.

According to yet another aspect of the device described above, each of the microfluidic channels is in fluidic communication with ports.

According to an alternative embodiment of the present invention, a device includes a first structure defining a first chamber, a second structure defining a second chamber; and a porous membrane located at an interface region between the first chamber and the second chamber. The membrane has a first side facing toward the first chamber and a second side facing toward the second chamber, the membrane separating the first chamber from the second chamber. The first side includes cells adhered thereto, the second side including a stimulus-responsive polymer gel thereon.

According to one aspect of the device described above, the first chamber is in the form of a first microchannel and the second chamber is in the form of a second microchannel.

According to another aspect of the device described above, the first chamber is a top chamber located above the porous membrane and the second chamber is a bottom chamber located below the porous membrane.

According to yet another aspect of the device described above, the second chamber is filled with the stimulus-responsive polymer gel.

According to yet another aspect of the device described above, the stimulus-responsive polymer gel is removable in response to an external stimulus.

According to yet another aspect of the device described above, the gel is fluid permeable.

According to yet another aspect of the device described above, the gel is water impermeable.

According to yet another aspect of the device described above, the cells include primary human airway epithelial cells.

According to yet another aspect of the device described above, the fluid-permeable, stimulus-responsive polymer gel is selected from a group consisting of Pluronic F127, Pluronic F68, poly(N-isopropylacrylamide), alginates, and a combination of two or more thereof.

According to another alternative embodiment of the present invention, a method is directed to preventing cell differentiation, and includes a) seeding viable cells on to a surface of a device under conditions such that at least a portion adheres thereon. The method also includes b) introducing a stimulus-responsive polymer gel on top of the cells under conditions such that differentiation of the cells is prevented and the cells remain viable.

According to one aspect of the method described above, the device is a Transwell device.

According to another aspect of the method described above, the device is a microfluidic device.

According to yet another aspect of the method described above, the microfluidic device includes a first microfluidic channel in fluidic communication with a second microfluidic channel.

According to yet another aspect of the method described above, the first microfluidic channel is separated from the second microfluidic channel by a porous membrane, the membrane having a first surface and a second surface.

According to yet another aspect of the method described above, the cells are adhered to the first surface of the membrane and the gel covers the adhered cells.

According to yet another aspect of the method described above, the method further includes c) feeding the cells with nutrients through the second surface of the membrane while the cells are covered on the first surface by the gel.

According to yet another aspect of the method described above, the cells are airway epithelial cells and the gel blocks the cells from air exposure, thereby preventing differentiation of the airway epithelial cells.

According to yet another aspect of the method described above, the method further includes d) removing the gel so that the cells are not prevented from differentiating.

According to yet another alternative embodiment of the present invention, a method is directed to preserving cells for storage or shipment, and includes a) seeding viable cells on to a surface of a device under conditions such that at least a portion adhere thereon. The method further includes b) introducing a stimulus-responsive polymer gel on top of the cells under conditions such the adhered cells remain viable, thereby preserving the cells.

According to one aspect of the method described above, the method further includes

c) shipping the cells.

According to another aspect of the method described above, the method further includes c) storing the cells.

According to yet another aspect of the method described above, the device is a Transwell device.

According to yet another aspect of the method described above, the device is a microfluidic device.

According to yet another aspect of the method described above, the microfluidic device includes a first microfluidic channel in fluidic communication with a second microfluidic channel.

According to yet another aspect of the method described above, the first microfluidic channel is separated from the second microfluidic channel by a porous membrane, the membrane having a first surface and a second surface.

According to yet another aspect of the method described above, the cells are adhered to the first surface of the membrane and the gel covers the adhered cells.

According to yet another aspect of the method described above, the method further includes c) removing the gel so that the cells are not covered by the gel.

According to yet another aspect of the method described above, the method further includes d) removing the gel so that the cells are not covered by the gel.

According to yet another alternative embodiment of the present invention, a method is directed to restricting the position of cells in a microfluidic device, the device including a first region and a second region. The method includes polymerizing a biopolymer material in the first region, seeding cells such that they attach to the second region but are prevented from attaching to the first region by the polymerized material, and after the seeding, depolymerizing and removing the biopolymer material from the first region.

According to one aspect of the method described above, the first region and second region are separated by a porous membrane, the porous membrane having top and bottom surfaces.

According to another aspect of the method described above, the method further includes imaging cell migration across the porous membrane.

According to yet another aspect of the method described above, the depolymerizing is in response to applying an external stimulus.

According to yet another aspect of the method described above, the external stimulus is one or more of a temperature application, a light application, a pH application, an enzyme application and a small molecule application.

According to yet another aspect of the method described above, the polymerizing of the biopolymer material includes incubation at a temperature of approximately 37° Celsius for approximately 30 minutes, the biopolymer material being a Pluronic F127 biopolymer.

According to yet another aspect of the method described above, the incubation results in a solidified material that blocks cell contact with the first region.

According to yet another aspect of the method described above, the first region includes a top microfluidic compartment selected from the group consisting of a microfluidic chamber or microfluidic channel.

According to yet another aspect of the method described above, the second region includes a bottom microfluidic compartment selected from the group consisting of a microfluidic chamber or microfluidic channel.

According to yet another aspect of the method described above, the first region includes a top surface of a membrane, or portion thereof.

According to yet another aspect of the method described above, the first region includes a bottom surface of a membrane, or portion thereof.

According to yet another aspect of the method described above, the microfluidic device is an organ-on-chip device.

According to yet another aspect of the method described above, the method further including seeding cells in the first region.

According to yet another aspect of the method described above, the cells in the first region are different from the cells in the second region.

According to yet another alternative embodiment of the present invention, a method is directed to restricting the position of cells in a microfluidic device, the device including a top compartment and a bottom compartment separated by a porous membrane. The method includes a) forming a gel in the top or bottom compartment or membrane, or portion thereof, and b) seeding cells on a region of the top or bottom compartments or membrane that is not blocked by the gel, thereby restricting the position of the cells in the microfluidic device.

According to one aspect of the method described above, the method further includes c) removing the gel from the microfluidic device.

According to another aspect of the method described above, the gel is removed by a method comprising depolymerizing the gel in response to applying an external stimulus.

According to yet another aspect of the method described above, the external stimulus is one or more of a temperature application, a light application, a pH application, an enzyme application and a small molecule application.

According to yet another aspect of the method described above, the forming of the gel in step a) includes polymerizing a biopolymer material.

According to yet another aspect of the method described above, the polymerizing of the biopolymer material includes incubation at a temperature of approximately 37° Celsius for approximately 30 minutes, the biopolymer material being a Pluronic F127 biopolymer.

According to yet another aspect of the method described above, the incubation results in a solidified material that blocks contact with the cells in a region of the microfluidic device.

According to yet another aspect of the method described above, the top microfluidic compartment is selected from the group consisting of a microfluidic chamber or microfluidic channel.

According to yet another aspect of the method described above, the bottom microfluidic compartment is selected from the group consisting of a microfluidic chamber or microfluidic channel.

According to yet another aspect of the method described above, the microfluidic device is an organ-on-chip device or a Transwell device.

According to yet another aspect of the method described above, the microfluidic device further includes ports in fluid communication with the top and bottom compartments and the seeding comprises introducing cells into the ports in a flow of culture media.

According to yet another alternative embodiment of the present invention, a method is directed to restricting the position of cells in a microfluidic device, the device including a porous membrane, the porous membrane including i) pores and ii) first and second sides. The method includes a) forming a gel in the first or second side of the membrane, or portion thereof, and b) seeding cells on a region of the membrane on the side opposite to the side including the gel under conditions such that the cells are prevented by the gel from moving through the pores, thereby restricting the position of the cells in the microfluidic device.

According to one aspect of the method described above, the method further includes c) removing the gel from the microfluidic device.

According to another aspect of the method described above, the gel is a water impermeable gel.

According to yet another aspect of the method described above, the gel is a calcium alginate gel and the gel is removed by washing out or chelating the calcium in the calcium alginate gel.

According to yet another aspect of the method described above, the microfluidic device is an organ-on-chip device or a Transwell device.

According to yet another aspect of the method described above, the gel is a collagen gel and the collagen gel is removed with an enzyme.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A is an exploded perspective view illustrating a device for simulating a function of a tissue, in accordance with one embodiment.

FIG. 1B is an assembled perspective view of the device shown in FIG. 1A.

FIG. 2A is a perspective view illustrating a device for simulating a function of a tissue, in accordance with another embodiment.

FIG. 2B shows the device of FIG. 2A with a biopolymer-filled bottom microchannel.

FIG. 2C shows the device of FIG. 2B with a cell-seeded top microchannel.

FIG. 2D shows the device of FIG. 2C with the biopolymer removed from the bottom microchannel.

FIG. 2E shows the device of FIG. 2D with a cell layer seeded in the bottom microchannel.

FIG. 3A shows a perspective view of an organ-on-chip (OOC) device, in accordance with yet another embodiment.

FIG. 3B shows an exploded perspective view of the OOC device of FIG. 3A.

FIG. 3C shows a cutaway perspective view with enlarged features of the OOC device of FIG. 3B.

FIG. 3D shows a cutaway side view with enlarged features of the OOC device of FIG. 3C.

FIG. 4 illustrates a system, in accordance with yet another embodiment, in which cells are seeded on top of a porous membrane.

FIG. 5 illustrates a system, in accordance with yet another embodiment, in which cells are submerged underneath a biopolymer.

FIG. 6A illustrates a TRANSWELL® insert system, in accordance with yet another embodiment.

FIG. 6B illustrates a biopolymer inserted in a bottom compartment of the system of FIG. 6A.

FIG. 6C illustrates cells seeded in a top compartment of the system of FIG. 6A.

FIG. 7A illustrates a TRANSWELL® insert system, in accordance with yet another embodiment.

FIG. 7B illustrates a biopolymer inserted in a top compartment of the system of FIG. 7A.

FIG. 7C illustrates cells seeded in a bottom compartment of the system of FIG. 7A.

FIG. 7D illustrates the biopolymer removed from the top compartment of the system of FIG. 7A.

FIG. 7E illustrates another cell type added to the top compartment of the system of FIG. 7A.

FIG. 7F illustrates a migration assay in the system of FIG. 7A.

FIG. 8 shows a timeline illustrating effects of delaying differentiation of cells.

FIG. 9A shows a microscopic image of epithelial morphology in a sample without polymer exposure.

FIG. 9B shows a microscopic image of epithelial morphology in a sample with polymer exposure.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

### Definitions

The term “microfluidic” as used herein relates to components where moving fluid is constrained in or directed through one or more chambers or channels wherein one or more dimensions are 1 millimeter (“mm”) or smaller (microscale). Microfluidic channels may be larger than microscale in one or more directions, though the channel(s) will be on the microscale in at least one direction. In some instances, the geometry of a microfluidic channel may be configured to control the fluid flow rate through the channel (e.g. increase channel height to reduce shear). Microfluidic channels can be formed of various geometries to facilitate a wide range of flow rates through the channels. Microfluidic devices include (but are not limited to) organ-on-chip devices and Transwell devices.

“Channels” are pathways (whether straight, curved, single, multiple, in a network, etc.) through a medium (e.g., silicon) that allow for movement of liquids and gasses. Channels, thus, can connect other components, i.e., keep components “in communication” and more particularly, “in fluidic communication,” and still more particularly, “in liquid communication.” Such components include, but are not limited to, liquid-intake ports and gas vents. Microchannels are channels with dimensions less than 1 mm and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source such as a fluid reservoir. Two components may be coupled to each other even though they are not in direct contact with each other. For example, two components may be coupled to each other through an intermediate component (e.g., tubing or other conduit).

Generally, according to one feature described below, undesirable cell migration is prevented when the migration occurs through a membrane with a pore size greater than 1 μm in diameter and when cells (such as endothelial cells) are cultured thereon in a microfluidic device, such as an organ-on-chip (“OOC”) device. To prevent cells from a first microchannel invading a second opposite microchannel, and, thus, interfering with plating or growth of tissue-specific cells in the second opposite microchannel, an approach includes seeding cells in the first microchannel of a microfluidic cell culture device (e.g., the OOC device) while the second opposite microchannel has a stimulus-responsive biocompatible polymer. Upon exposure to a first stimulus, the biocompatible polymer forms a gel to prevent cell invasion. The gel is readily dissolved or removed upon exposure to a second stimulus, when it is desired to plate another cell type in the second opposite microchannel. According to some examples, the gel includes a collagen type of gel, a water impermeable type of gel, and/or other types of gels.

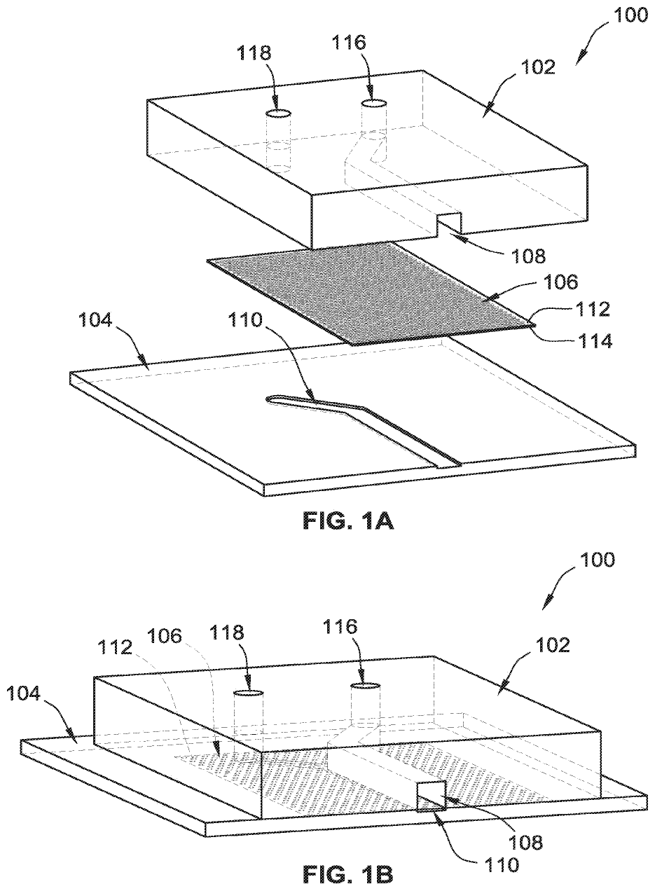

Referring to FIGS. 1A and 1B, an exemplary of a microfluidic device 100 is in the form of an OOC device that includes a top slab 102 separated from a bottom slab 104 by a porous membrane 106. The top slab 102 defines a first chamber 108 (which is illustrated in the form of a top microchannel), and the second slab 104 defines a second chamber 110 (which is illustrated in the form of a bottom microchannel). The porous membrane 106 is located at an interface region between the first chamber 108 and the second chamber 110, separating the two chambers 108, 110, and has a first side 112 facing toward the first chamber 108 and a second side 114 facing toward the second chamber 110. A first fluid inlet 116 is provided for fluid flow into the top microchannel 108, and a second fluid inlet 118 is provided for fluid flow into the bottom microchannel 110.

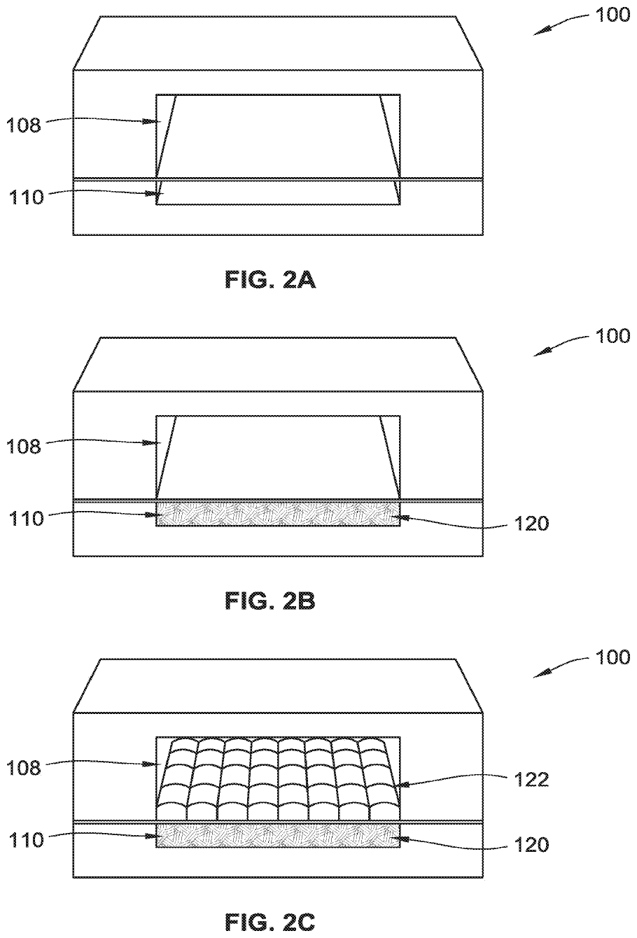

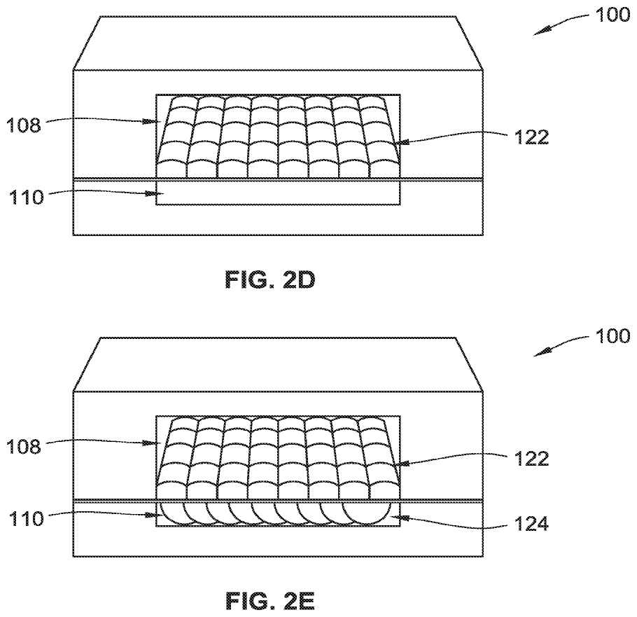

Referring generally to FIGS. 2A-2E, the culturing and prevention of undesirable cell migration is achieved, for example, in micro-, meso-, and/or macro-fluidic settings. Initially, in reference to FIG. 2A, the microfluidic device 100 is provided with the top and bottom microchannels 108, 110 empty (e.g., lacking any fluids, cultured cells, or gels). Then, in reference to FIG. 2B, the bottom microchannel 110 is temporally or temporarily filled with a biocompatible polymer gel 120 to prevent cell invasion. Referring to FIG. 2C, the top microchannel 108 is seeded with one or more cell layers 122. Referring to FIG. 2D, the polymer gel 120 is removed from the bottom microchannel 110. According to one example, the polymer gel 120 is removed with enzymes. The removal of the polymer gel 120 typically occurs when a user desires to plant another cell type in the bottom microchannel 110. Then, referring to FIG. 2E, the bottom microchannel 110 is seeded with one or more cell layers 124 (e.g., a blood-brain barrier or epithelial-endothelial co-culture systems).

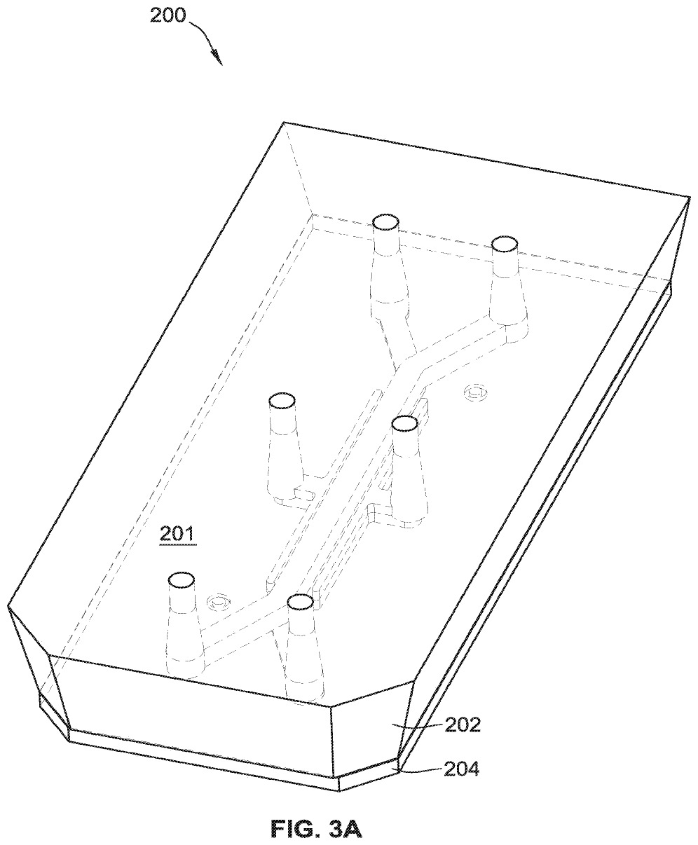

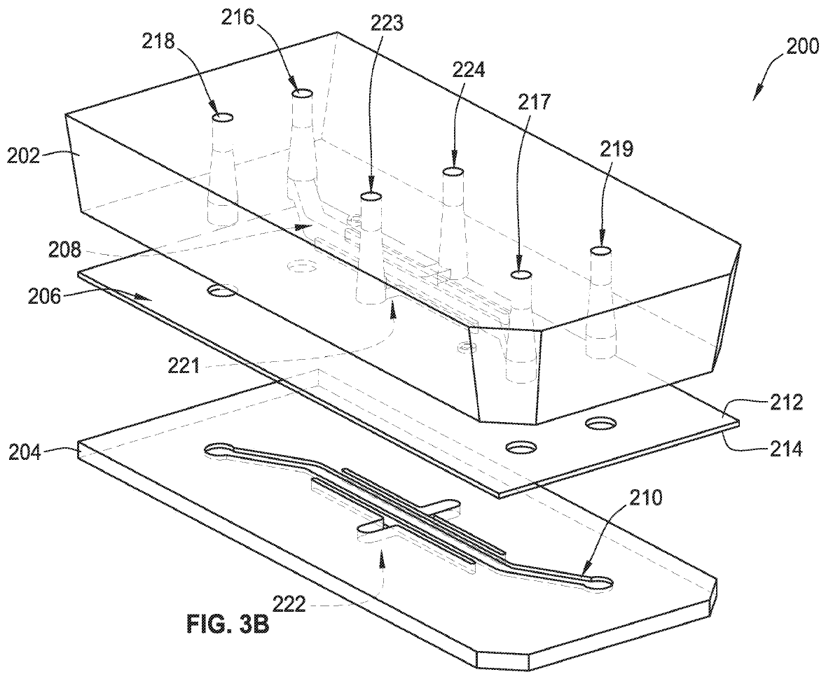

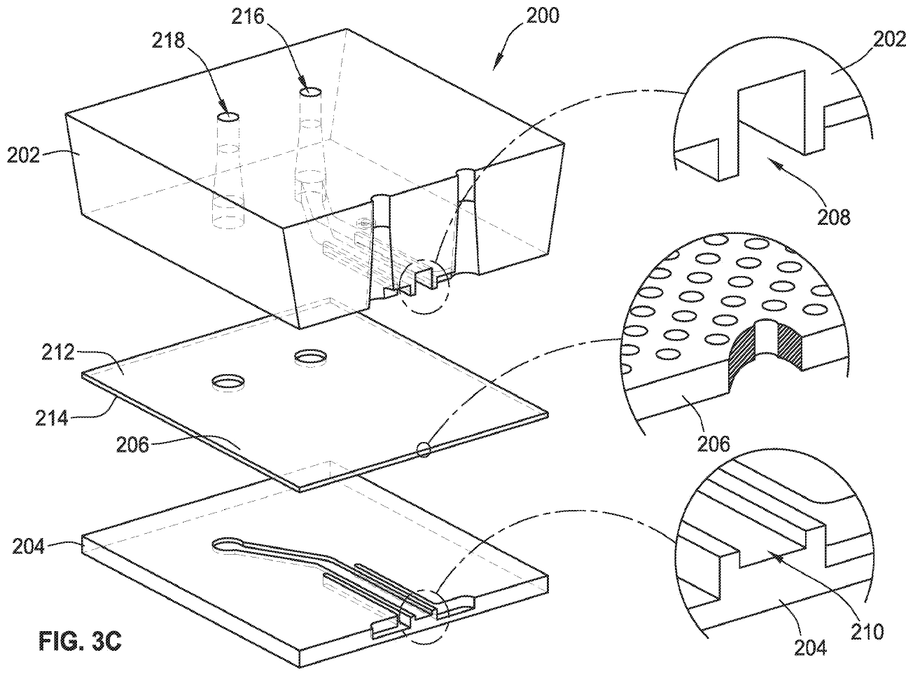

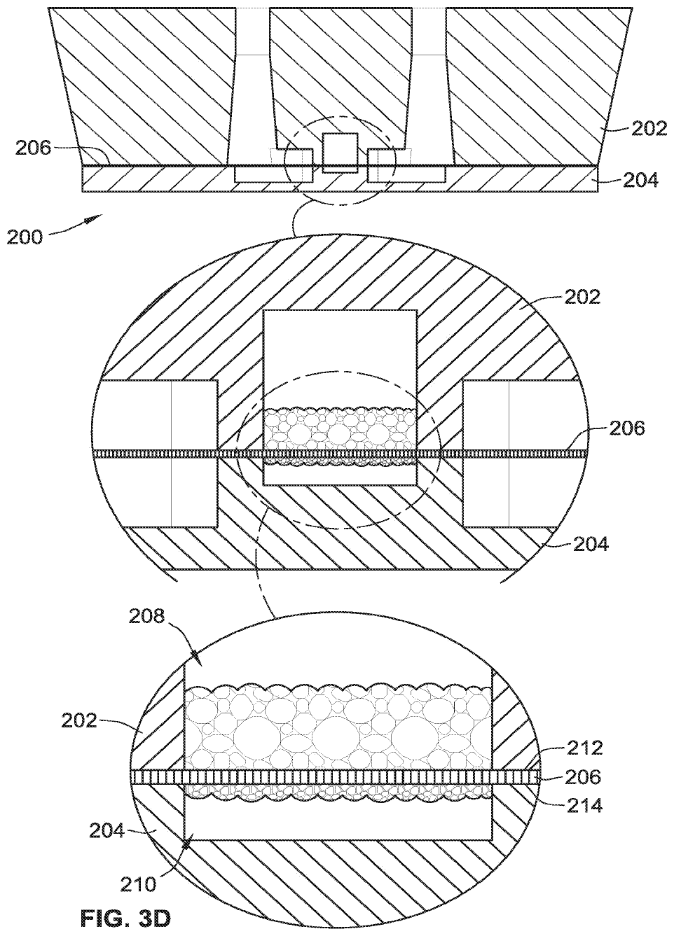

Referring generally to FIGS. 3A-3D, another exemplary embodiment is directed to an OOC device 200 that includes a body 201 with an upper body segment 202 and a lower body segment 204. The upper body segment 202 and the lower body segment 204 are preferably made of a polymeric material, such as PDMS, PMMA, polycarbonate, COP/CoC, polyurethane, or SBS/SEBS.

Referring specifically to FIGS. 3B and 3C, the OOC device 200 includes a porous membrane 206 that is located at an interface region between the upper body segment 202 and the lower body segment 204, separating a top microchannel 208 from a bottom microchannel 210. The membrane 206 has a first side 212 facing toward the top microchannel 208 and a second side 214 facing toward the bottom microchannel 210. A first fluid inlet 216 is provided for fluid flow into the top microchannel 208, and a second fluid inlet 218 is provided for fluid flow into the bottom microchannel 210.

Referring specifically to FIG. 3B, a first fluid outlet 217 is provided for fluid flow exiting the top microchannel 208, and a second fluid outlet 219 is provided for fluid flow exiting the bottom microchannel 210. Optionally, the OOC device 200 includes one or more additional top and bottom microchannels, such as cross-flow microchannels 221, 222, with respective third fluid inlet and outlet 223, 224.

Referring specifically to FIG. 3C, the membrane 206 includes a plurality of pores 226 through which cells are able to migrate between the top microchannel 208 and the bottom microchannel 210. The size of the pores 226 is selected in accordance with achieving a desired migration, simulating a specific function in vitro.

Referring specifically to FIG. 3D, the top microchannel 208 is filled at least in part with a biocompatible polymer gel 220, which is attached to the top surface 212 of the membrane 206. The bottom microchannel 210 includes a cell layer 222, which is seeded onto the bottom surface 214 of the membrane 206. While the polymer gel 220 is in place on the top surface 221 of the membrane 206, the polymer gel 220 prevents undesired or inadvertent migration of cells 222 from the bottom microchannel 210 into the top microchannel 208. When migration of cells 222 is desired, the polymer gel 220 is removed at least in part from the top microchannel 208.

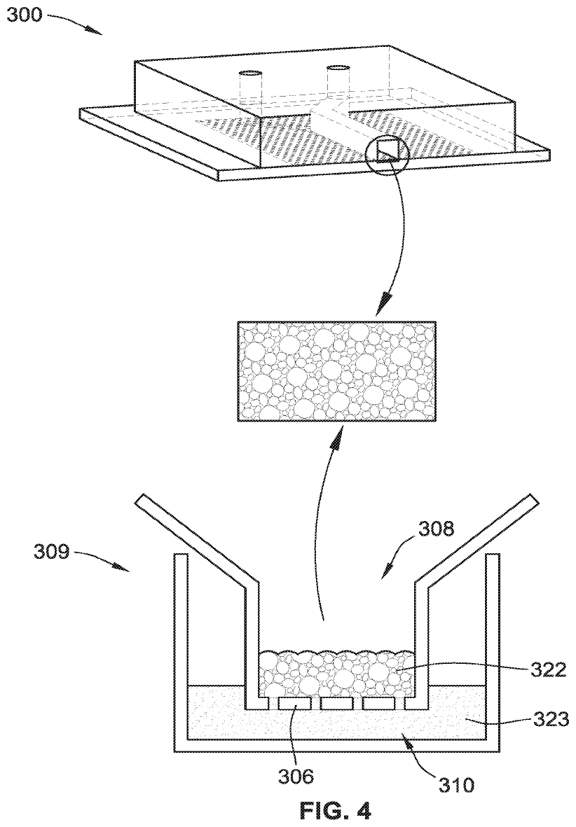

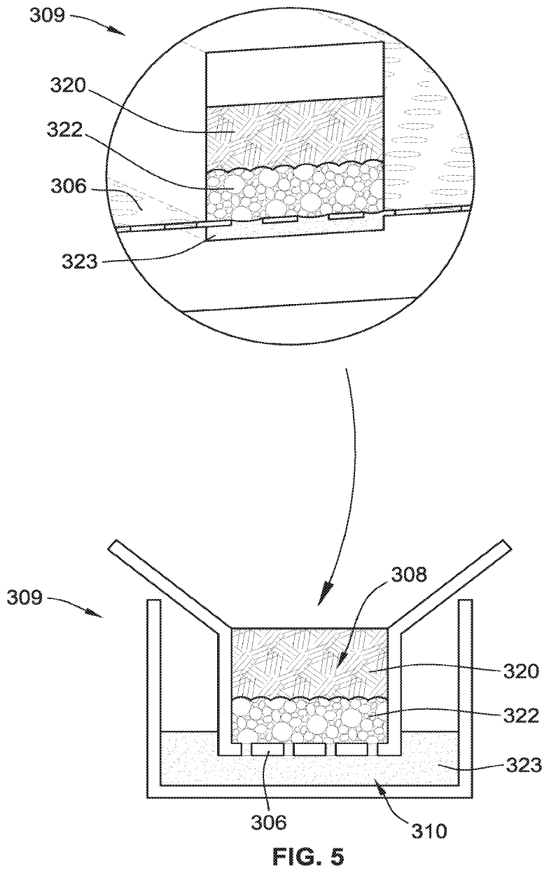

Referring generally to FIGS. 4 and 5, cells seeded on top of a porous membrane are submerged underneath a polymer gel (i.e., in the same microchannel as the cultured cells), according to an alternative embodiment. In specific reference to FIG. 4, a microfluidic device 300 includes a top microchannel 308, a porous membrane 306, and a bottom microchannel 310. Optionally, the microfluidic device 300, including one or more of its features, is similar to or identical to the microfluidic device 100 illustrated in FIGS. 1A and 1B. According to another optional embodiment, the microfluidic device 300 includes one or more features of a TRANSWELL® insert system 309, for a static cell culture.

The microfluidic device 300 includes primary human airway epithelial cells 322 that are seeded on top of the porous membrane 306, within the top microchannel 308. Additionally, the microfluidic device 300 includes a cell culture medium 323 in the bottom microchannel 310. Referring specifically to FIG. 5, the cells 322 are submerged underneath underneath a biopolymer gel 320. The cells 322 are kept submerged under the polymer without disrupting cell monolayer quality and the cell ability to differentiate in vitro. This aspect is helpful in serving at least two purposes: (1) to delay differentiation of desired cell type, and (2) to preserve and stabilize live cell layers 322 submerged under biopolymer gel 320 for shipment of OOC devices or for reducing the total amount of medium required. Thus, the microfluidic device 300 is beneficial in stabilizing cell cultures or replacing cell culture medium in cell-containing platforms for maintenance or shipment from one location to another. For example, by filling a microchannel with a biopolymer gel 320, the total amount of cell culture medium required is reduced.

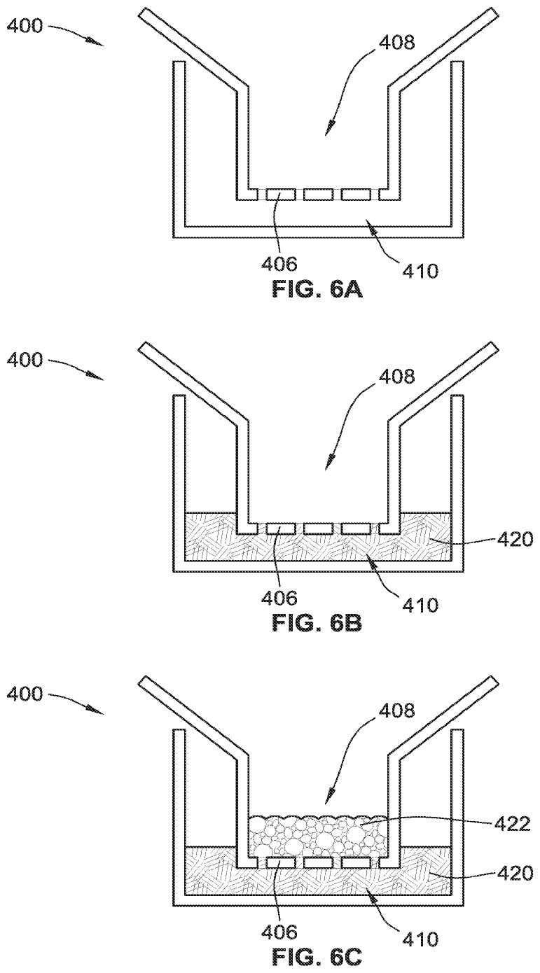

Referring generally to FIGS. 6A-6C, an exemplary method is illustrated in which cells are seed in a top compartment and a biopolymer gel is polymerized in a bottom compartment. Referring specifically to FIG. 6A, a microfluidic device 400 includes similar or identical features to the TRANSWELL® insert system 309 illustrated in FIGS. 4 and 5. By way of example, the microfluidic device 400 includes a top compartment 408 (e.g., an insert), a porous membrane 406, and a bottom compartment 410 (e.g., a well). Referring specifically to FIG. 6B, a biopolymer gel 420 is polymerized in the bottom compartment 410. Referring specifically to FIG. 6C, cells 422 are seeded on top of the membrane 406, in the top compartment 408. This approach is useful, for example, for assays such as immune cell chemotaxis assays in which a single endothelial layer is required on large-pore (e.g. 3-8 μm) membrane.

According to one feature, the microfluidic device 400 and the biopolymer gel 420 (e.g., filter-sterilized biocompatible polymer) are incubated under a condition that maintains the biopolymer gel 420 in a liquid form. For example, the microfluidic device and a biocompatible polymer Pluronic F127 are incubated at about 4° C. for about 30 minutes. The procedure is performed while the top compartment 408 (reserved for cell seeding) is clamped or blocked on both ends to prevent polymer leak. This step is carried out quickly, and, then, the polymer-filled microdevice is placed at a temperature selected for allowing the polymer to gel. For example, assuming that Pluronic F127 is the selected biocompatible polymer, the polymer-filled microdevice is placed in a 37° C. incubator for about 15 min to allow the biopolymer gel 420 to transition from liquid form to gel form. Meanwhile, a suspension of cells 422, e.g., endothelial cells such as human umbilical vein endothelial cells (“HUVECs”), is prepared at a desired concentration, with the cells 422 being added quickly to the top compartment 408 and allowed to adhere overnight. A culture medium of the cell-seeded compartment 408 is replaced with a fresh medium, e.g., by connecting the microfluidic device 400 to microfluidic flow. The microfluidic device 400 is continuously monitored for any sign of cell escape into the polymer-filled compartment 410.

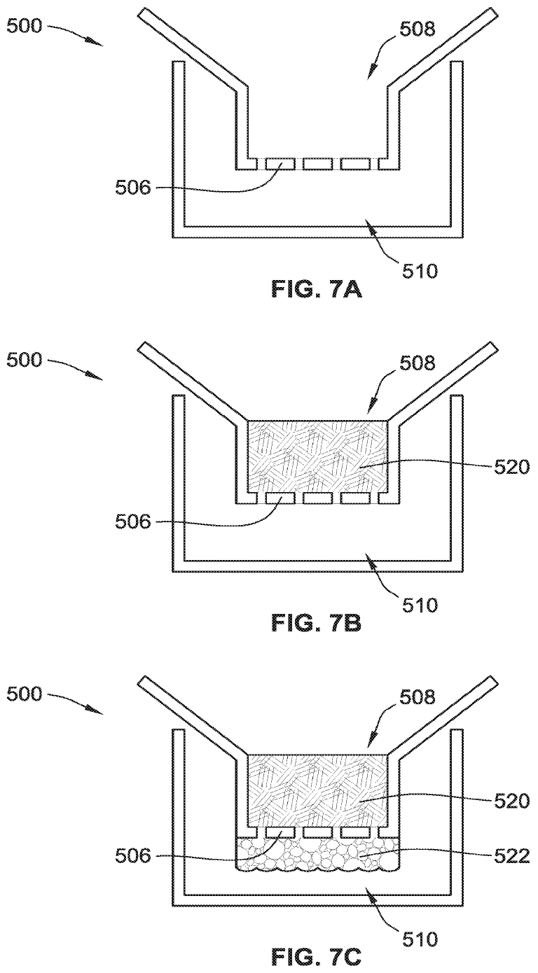

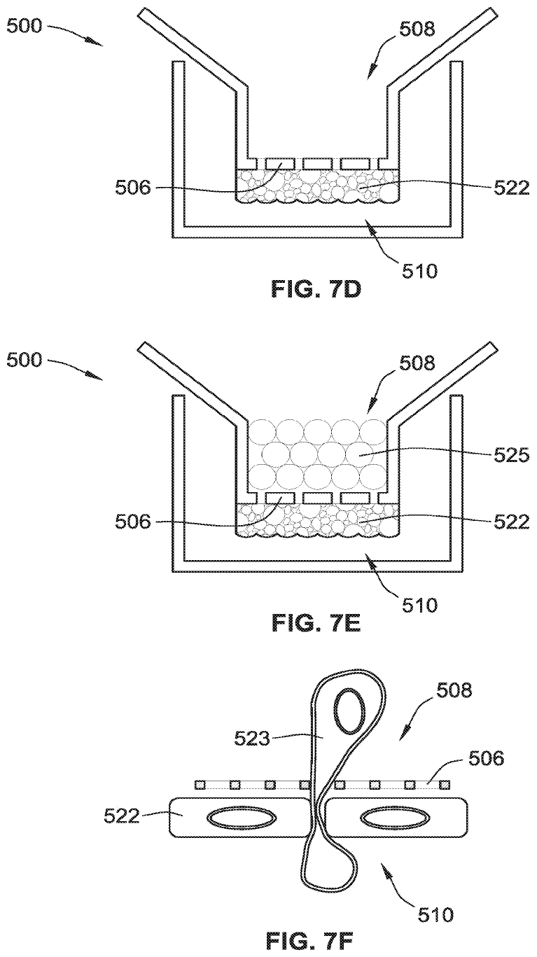

Referring generally to FIGS. 7A-7F, another exemplary method is illustrated in which a first type of cells migrates between compartments of a microfluidic device towards a second type of cells. Referring specifically to FIG. 7A, a microfluidic device 500 includes similar or identical features to the TRANSWELL® insert system 309 illustrated in FIGS. 4 and 5. By way of example, the microfluidic device 500 includes a top compartment 508, a porous membrane 506, and a bottom compartment 510. Referring specifically to FIG. 7B, a biopolymer gel 520 is polymerized in the top compartment 508. Referring specifically to FIG. 7C, a first type of cells 522 is seeded on the undersurface of the membrane 506, in the bottom compartment 510. Referring specifically to FIG. 7D, the biopolymer gel 520 is depolymerized and removed from the top compartment 508. Referring specifically to FIG. 7E, a second type of cells 525 is added to the top compartment 508. Referring specifically to FIG. 7F, a migration assay is performed to analyze (e.g., image) migration of one or more cells 523 of the first type of cells 522 that migrate through the membrane 506, from the bottom compartment 510 to the top compartment 508.

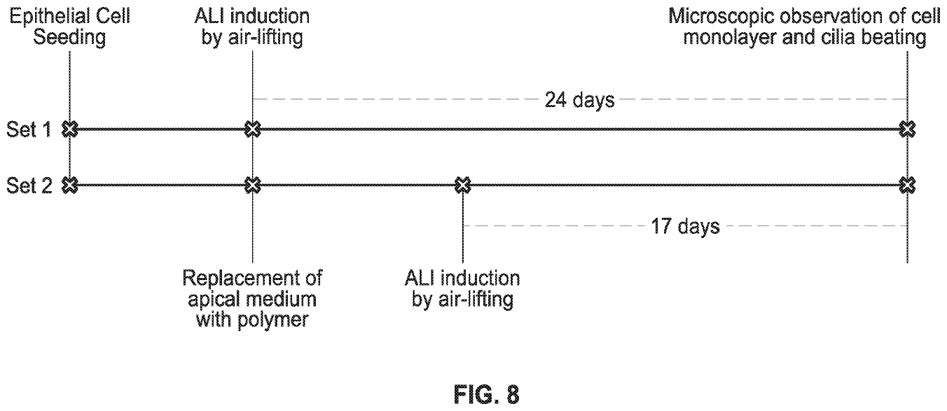

Referring to FIG. 8, a timeline illustrates delaying differentiation of cells in vitro without causing adverse effects on cell behavior and/or quality. The delaying of the cellular differentiation is for a desired period of time, e.g., for a number of days or a number of hours. To hold back the differentiation process, for example, cells are fed with nutrients and kept alive through one side (e.g., basal side) while the other side of the cells (e.g., apical side) is exposed to the biopolymer gel. By way of example, air exposure is a known inducer of cellular differentiation for cells like airway epithelial cells. Additionally, submerging the cells apically with polymer delays differentiation as long as a user intends.

By way of example, a first set of epithelial cells (Set 1) serves as a control group of samples (e.g., confluent epithelial monolayers) that are not exposed to a biopolymer gel. The first set of epithelial cells are directly taken to an air-liquid interface (“ALI”) 24 days prior to the microscopic observation of cell monolayer and cilia beating. ALI is a key driver of airway epithelial cell differentiation into ciliated cells.

In a second set of epithelial cells (Set 2), which is exposed to a biopolymer gel, a replacement of an apical medium with the biopolymer gel is also performed 24 days prior to the microscopic observation. Confluent human airway epithelial cells are submerged under the biopolymer gel for 7 days within a microfluidic device (e.g., an OOC device as described above). After the replacement of the apical medium with the biopolymer gel, the ALI induction is performed, later, only 17 days prior to the microscopic observation. Thus, differentiation is achieved by removing the biopolymer gel and introducing air into the microchannel to form the ALI. As discussed below, no adverse effects re observed in cell behavior and/or quality between the first set of epithelial cells and the second set of epithelial cells.

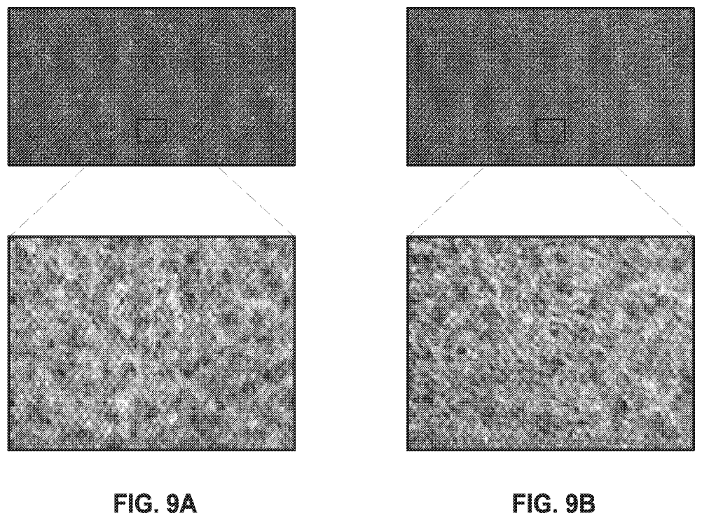

Referring generally to FIGS. 9A and 9B, sample microscopic images illustrate similar results in both sets of epithelial cells discussed above in reference to FIG. 8. Referring specifically to FIG. 9A, a phase-contrast microscopic image shows the first set of cells, which has not been exposed to the biopolymer gel, on day 24 (post-ALI) with a nice cobblestone packed epithelial morphology. Referring specifically to FIG. 9B, a phase-contrast microscopic images shows the second set of cells, which has been exposed to the biopolymer gel, on day 17 (post-ALI) with a similar nice cobblestone packed epithelial morphology. Cilia beating (10× slowed down) was measured for both sets of cells, with good and comparable measures in both sets of cells.

Thus, the cells that are overlaid with the biopolymer gel not only have a normal cobblestone packed epithelial monolayer (by benchmarking against and similar to polymer-untreated cells), but also exhibit an ability to differentiate into ciliated epithelium. However, the cell differentiation is delayed compared to the control cultures by approximately the number of days that the cells are kept covered with the polymer and held in a “suspended state.” This indicates that the cells are stabilized biologically by the biopolymer gel described herein to avoid unwanted differentiation, or potentially de-differentiation, during transport or long-term tissue maintenance. In addition, the extremely high viscosity of the gelled polymer prevents shear-induced damage to the tissue during transport and handling. This aspect is beneficial at least for shipping organs, tissues, or cells in a microfluidic device when they can be cultured in a suspended state. Upon receipt of the microfluidic device, the user removes the polymer, adds medium (or air), and reboots the differentiation process necessary to complete a desired study.

Exemplary biocompatible polymer gels for use in reference to any of the gels discussed above, include, but are not limited to the following:
Pluronic F127 (e.g., ˜25% w/v) and/or Pluronic F68, which both exhibit a reverse thermal gelling behavior, which gels at high temperatures (e.g., ˜20° C.) and melts at low temperatures (e.g., 4° C.);
Poly(N-isopropylacrylamide) (“PNIPAAm”) polymer, which is modified to gel at about 37° C. and to contract at a higher or lower temperature to facilitate gel removal; and
Alginates, which gels in the presence of calcium but dissolves when calcium chelators are added.

Pluronic F127 prevents endothelial cell escape and invasion into a second microchannel of an organ chip in an in vitro assay. Pluronic F127 is a triblock co-polymer that exhibits a reverse thermal gelling behavior in which, unlike most gels (e.g., agarose, gelatin, etc.), it solidifies at high temperatures (e.g., >20° C.) and melts at low temperatures (e.g., ˜4° C.). This behavior is applicable in many biomedical applications, such as areas of tissue engineering, drug delivery, and controlled compound release into tissue. Additional details regarding Pluronic F127 are described in “Applications of Thermo-Reversible Pluronic F-127 Gels in Pharmaceutical Formulations,” by J. J. Escobar-Chavez et al., published Nov. 27, 2006 (J. Pharm. Pharmaceut. Sci. 9 (3):339-358), which is incorporated herein by reference in its entirety. Pluronic F127 is also useful in other areas, such as 3D printing.

Pluronic F68 a similar behavior to Pluronic F127. Additional details regarding Pluronic F68 are described in “Rheological Properties of Thermo-Responsive Microemulsion-based Gels Formed by Pluronic F68,” by Zhao et al. (J. Chem. Pharm. Res., 2014, 6(7): 2067-2072), which is incorporated herein by reference in its entirety. Other polymers include any other polymer or materials with the ability to polymerize and depolymerize in a range of about 0-37° C. Such polymers or materials do not interfere with cell adhesion, growth, or differentiation (depending on the tested cell). Alternatively, the biocompatible polymer, according to some examples, is Pluronic F127 having 1-50% weight/volume (“w/v”), 15-30% w/v, or 25% w/v.

The PNIPAAm polymer provides the ability to be washed out of a microchannel of a microfluidic cell culture device (e.g., an organ chip) when cells (e.g., a different types of cells) are desired to be seeded in the microchannel. Alginates can also be used which can be gelled with calcium and dissolved with calcium chelators. Optionally, a low viscosity of the biocompatible polymer is at about 4° C. allows filling or emptying of very small microchannels and other features without affecting cells or devices due to pressure and shear. Optionally, yet, biocompatible polymers described herein are optically transparent and non-fluorescent in typical imaging wavelengths, unlike some other thermally-responsive gels (e.g. PNIPAAm).

According to one aspect, the stimulus responsive biocompatible polymer gels (which are not limited to thermally-responsive biocompatible polymer gels) are optionally or alternatively overlaid on top of a cultured cell monolayer. This approach, for example, delays initiation of a certain cell process (such as cell differentiation) until the organ chip is ready to use, and/or to protect submerged live cell layers from shear-induced damage during transport and handling.

By way of example, the biocompatible polymer gel is optionally selected from polymers that offer one or more of the following characteristics: (1) biocompatibility such that the biocompatible polymer gel does not interfere with culture, growth, and potentially differentiation of seeded cells; (2) the polymer gel does not readily leak into or block a cell-containing microchannel; and (3) the first microchannel is polymerized and de-polymerized using one or more stimuli that have minimal or no cytotoxicity or significant biological effects. For example, in reference to minimal biological effects, a change in temperature at an approximate range of 4°-37° C. is typically well-tolerated by many cells, but a large change in pH can have detrimental impact on the cells and cause cytotoxicity or cellular stress.

According to yet another aspect, the biocompatible polymer gel is non-biodegradable. For example, the biocompatible polymer gel does not include any extracellular matrix proteins that degrade by cells or that affect cell growth and/or functions.

According to yet another aspect, based on the polymer gel being of non-rigid form, a device is used for mechanical actuation or stimulation of the tissue without affecting the blocking polymer gel. Optionally, cell-compatible reversal and polymer removal processes avoid harsh chemical, ultraviolet (“UV”), and/or thermal treatments. The methods described herein are simple, fast, and sterile process-compatible methods. Optionally, yet, in addition to or instead of restricting cell position for a desirable period of time, biocompatible polymers can also be utilized to stabilize cell cultures in culture devices. For example, biocompatible polymers protect cells against movement-induced fluid shear forces and/or minimize the total amount of medium requirements. The protection and/or minimization features are beneficial for shipping or storage of tissues, organs, or cells in a microfluidic cell culture device (e.g., an organ chip) in a way that optimizes the respective structure and function.

According to yet another aspect, the biocompatible polymer transitions between liquid and gel states at about 25° C. for 25% w/v solutions. This biocompatible polymer enables removal at a cell-compatible temperature, such as 4° C. The biocompatible polymer is also optically clear and is non-autofluorescent, allowing the biocompatible polymer to be used without interfering with imaging applications. The biocompatible polymer does not prevent or negatively influence cell attachment and growth in a culture. For example, a monolayer becomes evident on day two of post seeding in a gel-free microchannel. The cell layer morphology is comparable to the morphology in chips that are seeded without polymer in the opposite microchannel. Importantly, no cell escape occurs to the unseeded microchannel.

In alternative embodiments, the biocompatible polymer undergoes polymerization-de-polymerization via a method other than one having a temperature change, as long as the method does not affect cell viability. Optionally or alternatively, any human and non-human (eukaryotic and prokaryotic) cell types, including but not limited to any human organ endothelial cells, epithelial cells, neurons, and/or immune cells, are used in reference to the microfluidic devices and methods described above.

In yet other alternative embodiments, the methods described herein are applied to cell patterning in general, using a polymer as a sacrificial barrier for a number of other applications, including 3D printing of tissues, cell co-culture, etc. Patterning, for example, is performed by filling microchannels, voids, or other geometric features, using stamping, printing, injection, spraying, or other methods for depositing materials in defined regions or patterns. In some applications, a reverse thermal gel polymer for controlling cell patterning. At least one microchannel of a cell culture microfluidic device is robustly blocked with an inert, stable, and biocompatible polymer.

In yet other alternative embodiments, the methods and devices described herein employ a biocompatible polymer that is modified with cell repulsing factors (which are not limited to semaphorins), cell anti-adhesion or adhesion moieties, growth factors, peptides, or any other molecule or multiple molecules. Optionally or alternatively, biocompatible polymer gels formed in a cell-unseeded microchannel contain agents and/or molecules, such as drugs, growth factors, and proteins for a controlled release. In further optional or alternative embodiments, a polymer-filled microchannel is washed and cellularized.

In yet other alternative embodiments, the methods and devices described herein are used for immune cell migration assays through an endothelial monolayer. For example, the endothelial monolayer is a bone marrow on a chip, a blood bone marrow barrier on a chip, or a lymph node on a chip. Optionally or alternatively, the methods described herein are automated or performed manually and/or are used with automated or manual OOC devices.

In yet other applications, a method is directed to visualizing cells at different locations of a cell culture device. For example, a biocompatible polymer gel is used to potentially change a refractive index to more easily visualize different cell layers in bi-, tri-, multi-cell co-culture systems. In another example, addition of a polymer gel to a top microchannel of a microfluidic culture allows imaging of a lower microchannel cell layer due to refractive index matching of the polymer and top cell layer.

Each of these embodiments and obvious variations thereof is contemplated as falling within the spirit and scope of the claimed invention, which is set forth in the following claims. Moreover, the present concepts expressly include any and all combinations and subcombinations of the preceding elements and aspects.

1-24. (canceled)

25. A method of preventing cell differentiation, comprising:
a) seeding viable cells on to a surface of a device under conditions such that at least a portion adheres thereon; and
b) introducing a stimulus-responsive polymer gel on top of the cells under conditions such that differentiation of the cells is prevented and the cells remain viable.

26-31. (canceled)

32. The method of claim 25, wherein the cells are airway epithelial cells and the gel blocks the cells from air exposure, thereby preventing differentiation of the airway epithelial cells.

33. The method of claim 32, further comprising c) removing the gel so that the cells are not prevented from differentiating.

34. A method of preserving cells for storage or shipment, comprising:
a) seeding viable cells on to a surface of a device under conditions such that at least a portion adhere thereon; and
b) introducing a stimulus-responsive polymer gel on top of the cells under conditions such the adhered cells remain viable, thereby preserving the cells.

35. The method of claim 34, further comprising c) shipping the cells.

36. The method of claim 34, further comprising c) storing the cells.

37-41. (canceled)

42. The method of claim 34, further comprising c) removing the gel so that the cells are not covered by the gel.

43. (canceled)

44. A method for restricting the position of cells in a microfluidic device, the device including a first region and a second region, the method comprising:
polymerizing a biopolymer material in the first region;
seeding cells such that they attach to the second region but are prevented from attaching to the first region by the polymerized material; and
after the seeding, depolymerizing and removing the biopolymer material from the first region.

45. The method of claim 44, wherein the first region and second region are separated by a porous membrane, the porous membrane having top and bottom surfaces.

46. The method of claim 45, further comprising imaging cell migration across the porous membrane.

47. The method of claim 44, wherein the depolymerizing is in response to applying an external stimulus.

48-57. (canceled)

58. A method for restricting the position of cells in a microfluidic device, the device including a first compartment and a second compartment separated by a porous membrane, wherein said porous membrane permits the passage of at least a first cell type, the method comprising:
a) forming a gel in the first compartment or portion thereof so as to block the passage of cells of said first type through at least a portion of said membrane; and
b) seeding cells of the said first cell type in a region of the second compartments; and
c) removing the gel from the microfluidic device.

59. The method of claim 58, further comprising d) culturing said cells.

60. The method of claim 59, wherein the gel is removed by a method comprising depolymerizing the gel in response to applying an external stimulus.

61. The method of claim 60, wherein the external stimulus is one or more of a temperature application, a light application, a pH application, an enzyme application and a small molecule application.

62. The method of claim 58, wherein the forming of the gel in step a) includes polymerizing a biopolymer material.

63-66. (canceled)

67. The method of claim 58, wherein the microfluidic device is an organ-on-chip device or a Transwell device.

68. The method of claim 58, wherein the microfluidic device further includes ports in fluid communication with the first and second compartments and the seeding comprises introducing cells into the ports in a flow of culture media.

69. A method for restricting the position of cells in a microfluidic device, the device including a porous membrane, the porous membrane including i) pores and ii) first and second sides, the method comprising:
a) forming a gel in the first or second side of the membrane, or portion thereof; and
b) seeding cells on a region of the membrane on the side opposite to the side comprising the gel under conditions such that the cells are prevented by the gel from moving through the pores, thereby restricting the position of the cells in the microfluidic device.

70. The method of claim 69, further comprising c) removing the gel from the microfluidic device.

71-74. (canceled)
